(19) United States
(12) Patent Application Publication
(10) Pub. No.: US 2023/0196823 A1
(43) Pub. Date: Jun. 22, 2023

(54) FINGER VEIN SENSORS

(71) Applicant: NANJING EASTHOUSE ELECTRICAL CO., LTD., Nanjing (CN)

(72) Inventor: HAO MIN, NANJING (CN)

(73) Assignee: NANJING EASTHOUSE ELECTRICAL CO., LTD., Nanjing (CN)

(21) Appl. No.: 17/552,618

(22) Filed: Dec. 16, 2021

(51) Int. Cl. G06V 40/145; G16H 30/20; A61B 5/1171; G06V 10/141

(52) U.S. Cl. CPC G06V 40/145; G16H 30/20; A61B 5/1171; G06V 10/141

(57) ABSTRACT

The present disclosure relates to several types of finger vein sensor. In certain embodiments, the finger vein sensor includes: an image sensor, and an infrared light source. Image sensor captures infrared image of finger vein pattern of a finger of a target human. The image sensor faces down and is positioned at top of finger vein sensor. The infrared light source may include a predetermined number of infrared light-emitting diodes (LED), and they are arranged in one or more rows and one or more columns and positioned at bottom of finger vein sensor. The finger is positioned between infrared light source and image sensor. The infrared light from the infrared light source irradiates the finger vertically from the bottom to generate the infrared image of finger vein pattern of the finger on the image sensor, and the image sensor captures the infrared image of finger vein pattern of the finger.

### FIELD

The present disclosure generally relates to user authentication, and more particularly to several finger vein sensors that provide better finger vein pattern, and are contamination resistant.

### BACKGROUND

Finger vein sensors have been widely used for user authentication. However, the conventional finger vein sensors have some issues that need to be improved. As shown in related art FIG. 10A and FIG. 10B, the finger vein sensor 1000 includes a sensor body 1001, a finger vein pattern sensing surface 1005, and an infrared light-emitting diode (LED) 1009. The infrared light-emitting diode (LED) 1009 irradiates infrared light on a finger 1007 and generates finger vein pattern on the finger vein pattern sensing surface 1005. The finger vein pattern sensing surface 1005 captures the finger vein pattern for user authentication. As shown in FIG. 10A and FIG. 10B, the finger vein pattern sensing surface 1005 is relatively small compared to the size of the finger 1007. Therefore, only a small portion of the finger vein pattern is captured for user authentication. The finger 1007 often touches the finger vein pattern sensing surface 1005, and any contamination on the surface of the finger 1007 may distort the finger vein pattern of the finger 1007 captured, which may cause authentication errors. Additionally, the sensor body 1001 defines a small space 1003 above the finger vein pattern sensing surface 1005. If the space 1003 has water on it, the finger vein sensor 1000 will fail. Therefore, the conventional finger vein sensors are widely used only in indoor applications.

Therefore, heretofore unaddressed needs still exist in the art to address the aforementioned deficiencies and inadequacies.

### SUMMARY

In one aspect, the present disclosure relates to a finger vein sensor. In certain embodiments, the finger vein sensor includes: an image sensor, an infrared light source, and a finger vein sensor controller. The image sensor is horizontally positioned at an upper end of the finger vein sensor. The image sensor faces down in vertical direction to capture at least one infrared image of finger vein pattern of a finger of a target human. The infrared light source is positioned at a lower end of the finger vein sensor. The infrared light source faces up and shines infrared light upward in vertical direction. The finger vein sensor controller includes a processor, and a non-volatile memory storing an operating system and computer executable instructions. The computer executable instructions include: an infrared light source control module, a finger vein verification module, an image/parameter storage module, an image processing module, an image sensor control module, and a finger vein sensor power module.

In certain embodiments, when executed at the processor, the computer executable instructions cause the processor to perform one or more of operations:

detecting, by the image sensor, that the finger is placed between the infrared light source and the image sensor;

irradiating, by the infrared light source from the lower end of the finger vein sensor, the infrared light through the finger to generate an infrared image of finger vein pattern of the finger on the image sensor;

detecting, by the image sensor, clarity, brightness, contrast, noise distortion and blurring distortion of the finger vein pattern image of the finger of the target human;

adjusting, by the infrared light source control module, a plurality of parameters of the infrared light source, until each of the clarity, the brightness, the contrast, the noise distortion, and the blurring distortion of the finger vein pattern image of the finger reaches a corresponding predetermined level; and

capturing, by the image sensor, the infrared image of finger vein pattern of the finger, and storing the captured infrared image of finger vein pattern of the finger into the image/parameter storage module.

In certain embodiments, the infrared light source includes a group of infrared light-emitting diodes (LED), a group of infrared light bulbs, and/or any other infrared light sources. The group of infrared LED and the group of infrared light bulbs are arranged in one or more rows and one or more columns.

In certain embodiments, the finger vein sensor includes a lens and an infrared filter. The lens is positioned between the finger and the image sensor. The infrared filter is positioned between the lens and the image sensor for improving quality of the infrared image of finger vein pattern of the finger.

In certain embodiments, the finger vein sensor further includes a transparent finger resting surface for resting the finger on the transparent finger resting surface. The finger vein sensor further includes a finger vein sensor enclosure. The finger vein sensor enclosure includes a lower compartment for positioning the infrared light source and an upper compartment for positioning the image sensor and the lens. An upper surface of the lower compartment forms the transparent finger resting surface. A lower surface of the upper compartment forms a transparent surface.

In another aspect, the present disclosure relates to a finger vein sensor. In certain embodiments, the finger vein sensor includes an image sensor, an infrared light source, an optical reflector, and a finger vein sensor controller. The image sensor is vertically positioned in a user-facing side of an upper end of the finger vein sensor. The image sensor faces center of the finger vein sensor in horizontal direction to capture at least one infrared image of finger vein pattern of a finger of a target human. The infrared light source is positioned at a lower end of the finger vein sensor. The infrared light source faces upward and shines infrared light upward in vertical direction. The optical reflector is positioned in an optical path between the image sensor and the infrared light source. The optical reflector reflects a vertically oriented infrared image of finger vein pattern of the finger to the horizontally oriented image sensor.

In certain embodiments, the finger vein sensor controller includes a processor, and a non-volatile memory. The non-volatile memory stores an operating system and computer executable instructions. The computer executable instructions include: an infrared light source control module, a finger vein verification module, an image/parameter storage module, an image processing module, an image sensor control module, and a finger vein sensor power module. When executed at the processor, the computer executable instructions cause the processor to perform one or more of operations:

detecting, by the image sensor, that the finger is placed between the infrared light source and the optical reflector;

irradiating, by the infrared light source from the lower end of the finger vein sensor, the infrared light through the finger to generate an infrared image of finger vein pattern of the finger on the image sensor through the optical reflector;

detecting, by the image sensor, clarity, brightness, contrast, noise distortion and blurring distortion of the finger vein pattern image of the finger of the target human;

adjusting, by the infrared light source control module, a plurality of parameters of the infrared light source, until each of the clarity, the brightness, the contrast, the noise distortion, and the blurring distortion of the finger vein pattern image of the finger reaches a corresponding predetermined level; and

capturing, by the image sensor, the infrared image of finger vein pattern of the finger, and storing the captured infrared image of finger vein pattern of the finger into the image/parameter storage module.

In certain embodiments, the infrared light source includes a group of infrared light-emitting diodes (LED), a group of infrared light bulbs, and/or any other infrared light sources. The group of infrared LED and the group of infrared light bulbs are arranged in one or more rows and one or more columns.

In certain embodiments, the optical reflector includes a reflecting mirror, a triangular reflecting glass, or any other optical reflecting devices.

In certain embodiments, the finger vein sensor includes a lens and an infrared filter. The lens is positioned between the optical reflector and the image sensor. The infrared filter is positioned between the lens and the image sensor for improving quality of the infrared image of finger vein pattern of the finger.

In certain embodiments, the finger vein sensor further includes a transparent finger resting surface for resting the finger on the transparent finger resting surface. The finger vein sensor further includes a finger vein sensor enclosure. The finger vein sensor enclosure includes a lower compartment for positioning the infrared light source and an upper compartment for positioning the image sensor and the lens. An upper surface of the lower compartment forms the transparent finger resting surface. A lower surface of the upper compartment forms a transparent surface.

In yet another aspect, the present disclosure relates to a finger vein sensor. In certain embodiments, the finger vein sensor includes: an image sensor, an infrared light source, an optical reflector, and a finger vein sensor controller. In one embodiment, the image sensor is vertically positioned in a left side of an upper end of the finger vein sensor. In another embodiment, the image sensor is vertically positioned in a right side of the upper end of the finger vein sensor. The image sensor faces a center of the finger vein sensor in horizontal direction to capture at least one infrared image of finger vein pattern of a finger of a target human through the optical reflector.

In certain embodiments, the infrared light source is positioned at a lower end of the finger vein sensor. The infrared light source faces upward and shines infrared light upward in vertical direction. In certain embodiments, the infrared light source includes a group of infrared light-emitting diodes (LED), a group of infrared light bulbs, and/or any other infrared light sources. The group of infrared LED and the group of infrared light bulbs are arranged in one or more rows and one or more columns.

In certain embodiments, the optical reflector is positioned in an optical path between the image sensor and the infrared light source, wherein the optical reflector reflects a vertically oriented infrared image of finger vein pattern of the finger to the horizontally oriented image sensor. In certain embodiments, the optical reflector includes a reflecting mirror, a triangular reflecting glass, or any other optical reflecting devices.

In certain embodiments, the finger vein sensor controller includes a processor, and a non-volatile memory. The non-volatile memory stores an operating system and computer executable instructions. The computer executable instructions include: an infrared light source control module, a finger vein verification module, an image/parameter storage module, an image processing module, an image sensor control module, and a finger vein sensor power module. When executed at the processor, the computer executable instructions cause the processor to perform one or more of operations:

detecting, by the image sensor, that the finger is placed between the infrared light source and the optical reflector;

irradiating, by the infrared light source from the lower end of the finger vein sensor, the infrared light through the finger to generate an infrared image of finger vein pattern of the finger on the image sensor through the optical reflector;

detecting, by the image sensor, clarity, brightness, contrast, noise distortion and blurring distortion of the finger vein pattern image of the finger of the target human;

adjusting, by the infrared light source control module, a plurality of parameters of the infrared light source, until each of the clarity, the brightness, the contrast, the noise distortion, and the blurring distortion of the finger vein pattern image of the finger reaches a corresponding predetermined level; and

capturing, by the image sensor, the infrared image of finger vein pattern of the finger, and storing the captured infrared image of finger vein pattern of the finger into the image/parameter storage module.

In certain embodiments, the finger vein sensor further includes: a lens and an infrared filter. The lens is positioned between the optical reflector and the image sensor. The infrared filter is positioned between the lens and the image sensor for improving quality of the infrared image of finger vein pattern of the finger.

In certain embodiments, the finger vein sensor further includes: a finger vein sensor enclosure. The finger vein sensor enclosure includes a lower compartment and an upper compartment. The infrared light source is positioned in the low compartment, and the image sensor and the lens are positioned in the upper compartment. An upper surface of the lower compartment forms a transparent finger resting surface. A lower surface of the upper compartment forms a transparent surface.

These and other aspects of the present disclosure will become apparent from the following description of the preferred embodiment taken in conjunction with the following drawings, although variations and modifications therein may be effected without departing from the spirit and scope of the novel concepts of the disclosure.

### BRIEF DESCRIPTION OF THE DRAWINGS

The accompanying drawings illustrate one or more embodiments of the present disclosure, and features and benefits thereof, and together with the written description, serve to explain the principles of the present invention. Wherever possible, the same reference numbers are used throughout the drawings to refer to the same or like elements of an embodiment, and wherein:

FIG. 1 illustrates a configuration of a finger vein sensor 100 having an image sensor positioned on a top end of a finger vein sensor enclosure according to certain embodiments of the present disclosure;

FIG. 2 shows a block diagram of a finger vein sensor controller 200 of a finger vein sensor 100 according to certain embodiments of the present disclosure;

FIG. 3 illustrates a configuration of a finger vein sensor 100 having an image sensor positioned on a front facing side of the finger vein sensor enclosure and a reflecting mirror as an optical reflector according to certain embodiments of the present disclosure;

FIG. 4 illustrates a configuration of a finger vein sensor 100 having an image sensor positioned on a front facing side of the finger vein sensor enclosure and a triangular reflecting glass as an optical reflector according to certain embodiments of the present disclosure;

FIG. 5 illustrates a configuration of a finger vein sensor 100 having an image sensor positioned on a right side of the finger vein sensor enclosure and a reflecting mirror as an optical reflector according to certain embodiments of the present disclosure;

FIG. 6 illustrates a configuration of a finger vein sensor 100 having an image sensor positioned on the right side of the finger vein sensor enclosure and the reflecting mirror as the optical reflector according to certain embodiments of the present disclosure;

FIG. 7 illustrates a configuration of a finger vein sensor 100 having an image sensor positioned on a left side of the finger vein sensor enclosure and a triangular reflecting glass as an optical reflector according to certain embodiments of the present disclosure;

FIG. 8 shows a sectional view of a finger vein sensor 100 having an image sensor positioned on the left side of the finger vein sensor enclosure and the triangular reflecting glass as the optical reflector according to certain embodiments of the present disclosure;

FIG. 9 shows a flowchart of the finger vein sensor controller 200 to detect and capture one or more of the finger vein pattern images according to certain embodiments of the present disclosure; and

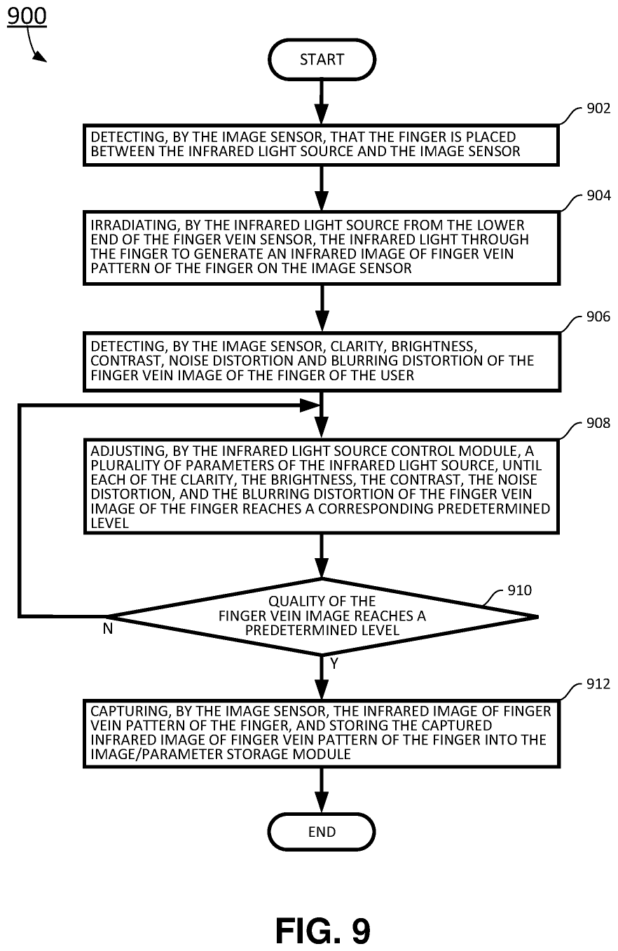

FIG. 10A shows a front view of a conventional finger vein sensor 1000, and FIG. 10B shows a side sectional view of the conventional finger vein sensor 1000.

### DETAILED DESCRIPTION

The present disclosure is more particularly described in the following examples that are intended as illustrative only since numerous modifications and variations therein will be apparent to those skilled in the art. Various embodiments of the disclosure are now described in detail. Referring to the drawings, like numbers, if any, indicate like components throughout the views. As used in the description herein and throughout the claims that follow, the meaning of “a”, “an”, and “the” includes plural reference unless the context clearly dictates otherwise. Also, as used in the description herein and throughout the claims that follow, the meaning of “in” includes “in” and “on” unless the context clearly dictates otherwise. Moreover, titles or subtitles may be used in the specification for the convenience of a reader, which shall have no influence on the scope of the present disclosure. Additionally, some terms used in this specification are more specifically defined below.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the disclosure or of any exemplified term. Likewise, the disclosure is not limited to various embodiments given in this specification.

Unless otherwise defined, all technical and scientific terms used herein have the same meaning as commonly understood by one of ordinary skill in the art to which this

disclosure pertains. In the case of conflict, the present document, including definitions will control.

As used herein, “around”, “about” or “approximately” shall generally mean within 20 percent, preferably within 10 percent, and more preferably within 5 percent of a

given value or range. Numerical quantities given herein are approximate, meaning that the term “around”, “about” or “approximately” can be inferred if not expressly stated.

As used herein, “plurality” means two or more.

As used herein, the terms “comprising,” “including,” “carrying,” “having,” “containing,” “involving,” and the like are to be understood to be open-ended, i.e., to mean including but not limited to.

As used herein, the phrase at least one of A, B, and C should be construed to mean a logical (A or B or C), using a non-exclusive logical OR. It should be understood that one or more steps within a method may be executed in different order (or conconventionally) without altering the principles of the present disclosure.

As used herein, the term module may refer to, be part of, or include an Application Specific Integrated Circuit (ASIC); an electronic circuit; a combinational logic circuit; a field programmable gate array (FPGA); an electronic key processor (shared, dedicated, or group) that executes code; other suitable hardware components that provide the described functionality; or a combination of some or all of the above, such as in a system-on-chip. The term module may include memory (shared, dedicated, or group) that stores code executed by the electronic key processor.

The term code, as used above, may include software, firmware, and/or microcode, and may refer to programs, routines, functions, classes, and/or objects. The term shared, as used above, means that some or all code from multiple modules may be executed using a single (shared) electronic key processor. In addition, some or all code from multiple modules may be stored by a single (shared) memory. The term group, as used above, means that some or all code from a single module may be executed using a group of electronic key processors. In addition, some or all code from a single module may be stored using a group of memories.

The apparatuses and methods described herein may be implemented by one or more computer programs executed by one or more electronic key processors. The computer programs include electronic key processor-executable instructions that are stored on a non-transitory tangible computer readable medium. The computer programs may also include stored data. Non-limiting examples of the non-transitory tangible computer readable medium are nonvolatile memory, magnetic storage, and optical storage.

The present disclosure will now be described more fully hereinafter with reference to the accompanying drawings, in which embodiments of the disclosure are shown. This disclosure may, however, be embodied in many different forms and should not be construed as limited to the embodiments set forth herein; rather, these embodiments are provided so that this disclosure will be thorough and complete, and will fully convey the scope of the disclosure to those skilled in the art. Like numbers refer to like elements throughout.

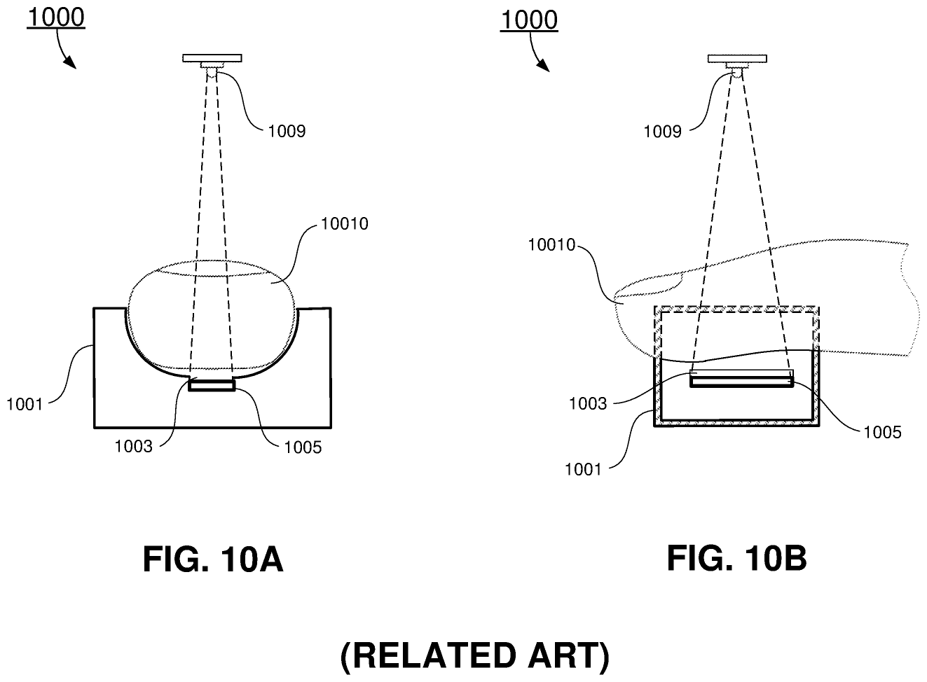

As shown in related art FIG. 10A and FIG. 10B, the conventional finger vein sensor 1000 has, among other things, following disadvantages. The finger vein pattern sensing surface 1005 is relatively small compared to the size of the finger 10010. Therefore, only a small portion of the finger vein pattern is captured for user authentication. The finger 10010 often touches the finger vein pattern sensing surface 1005, and any contamination on the surface of the finger 407 may distort the finger vein pattern of the finger 10010 captured, which may cause authentication errors. Additionally, the sensor body 1001 defines a small space 1003 above the finger vein pattern sensing surface 405. If the space 1003 has water in it, the finger vein sensor 1000 will fail. The present disclosure discloses several new improvements that will increase the size of the finger vein pattern images, that will eliminate authentication error caused by sensor surface contamination, and that will prevent authentication failures caused by moisture or water on the sensor surface.

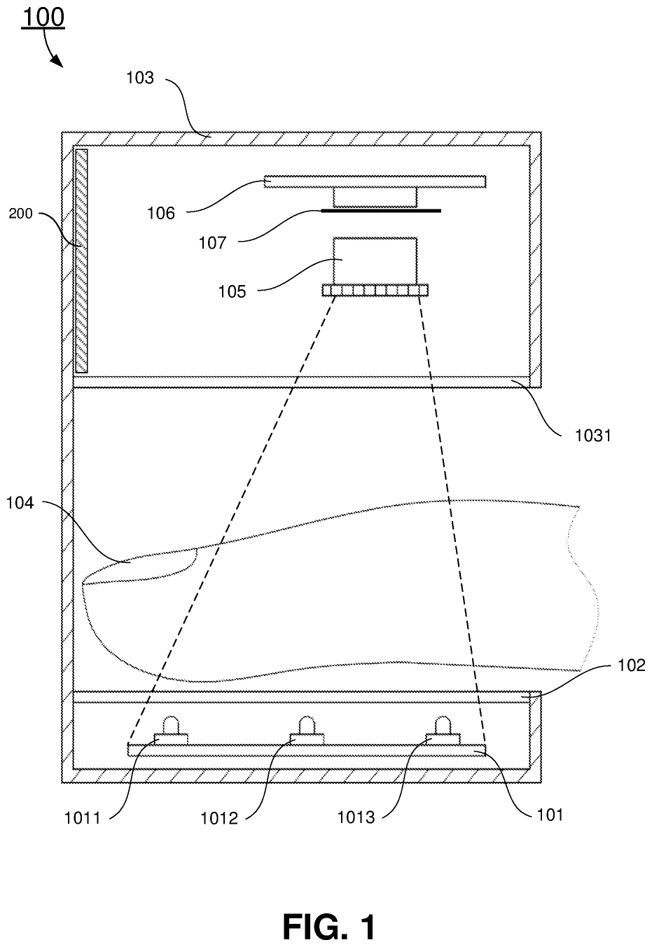

In one aspect, as shown in FIG. 1, the present disclosure relates to a finger vein sensor 100. In certain embodiments, the finger vein sensor 100 includes: an image sensor 106, an infrared light source 101, and a finger vein sensor controller 200. The image sensor 106 is horizontally positioned at an upper end of the finger vein sensor 100. The image sensor 106 faces down in vertical direction to capture at least one infrared image of finger vein pattern of a finger 104 of a target human. The infrared light source 101 is positioned at a lower end of the finger vein sensor 100. The infrared light source 101 faces up and shines infrared light upward in vertical direction.

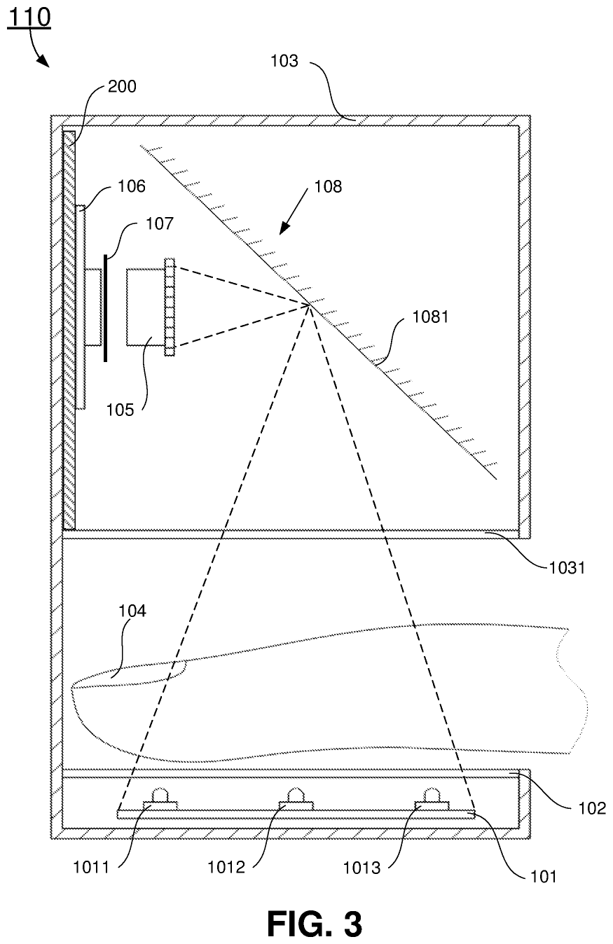

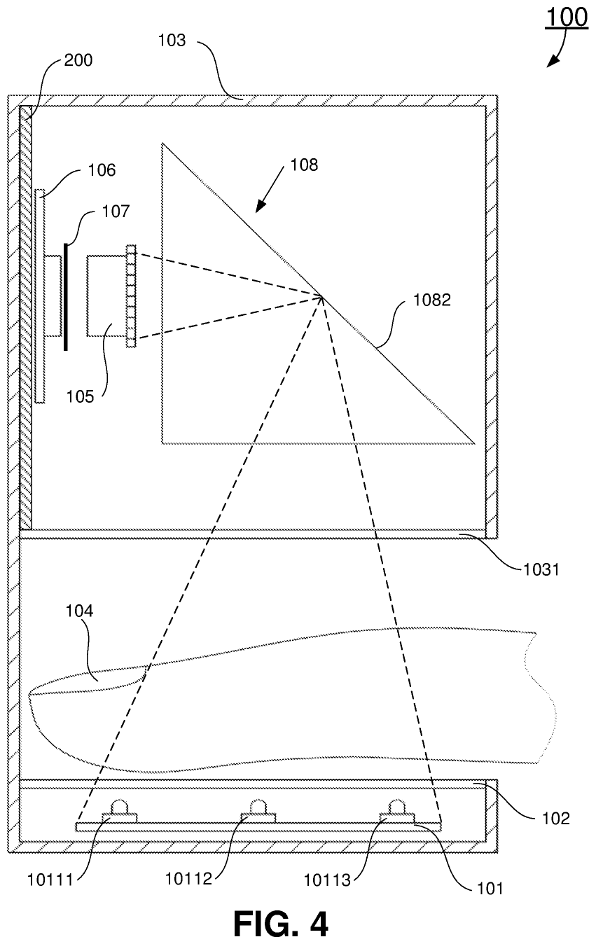

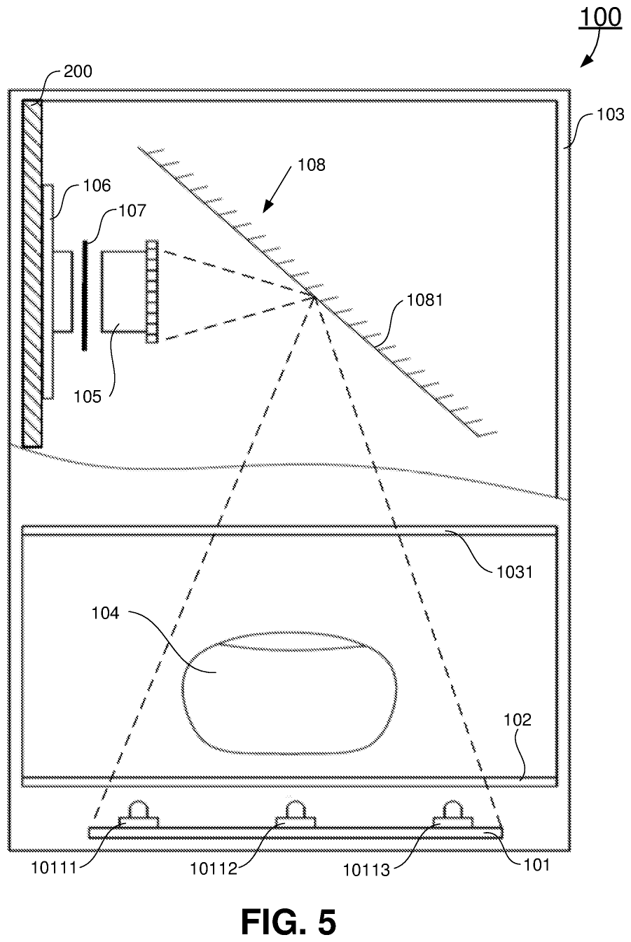

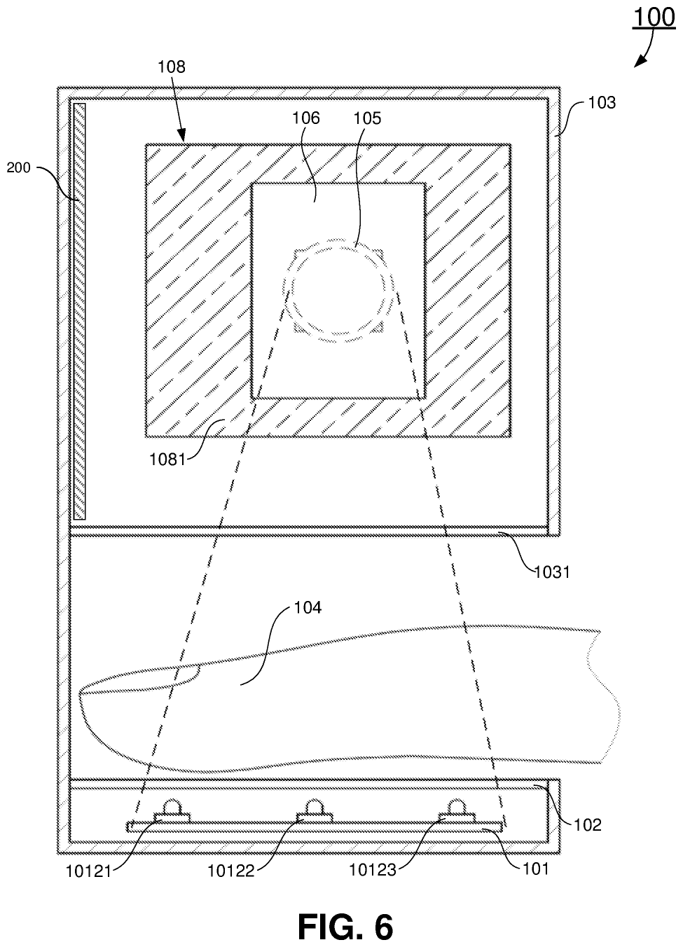

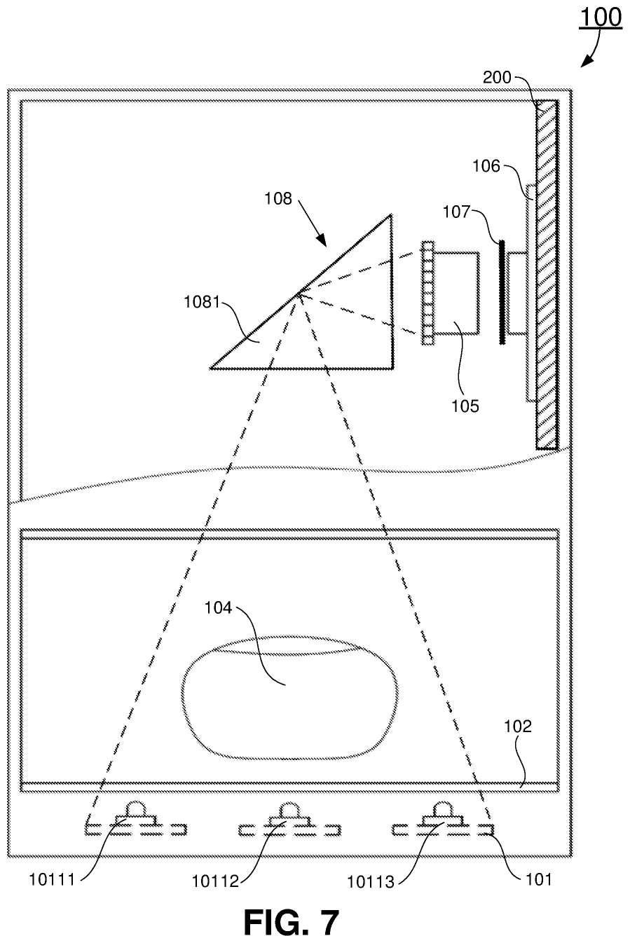

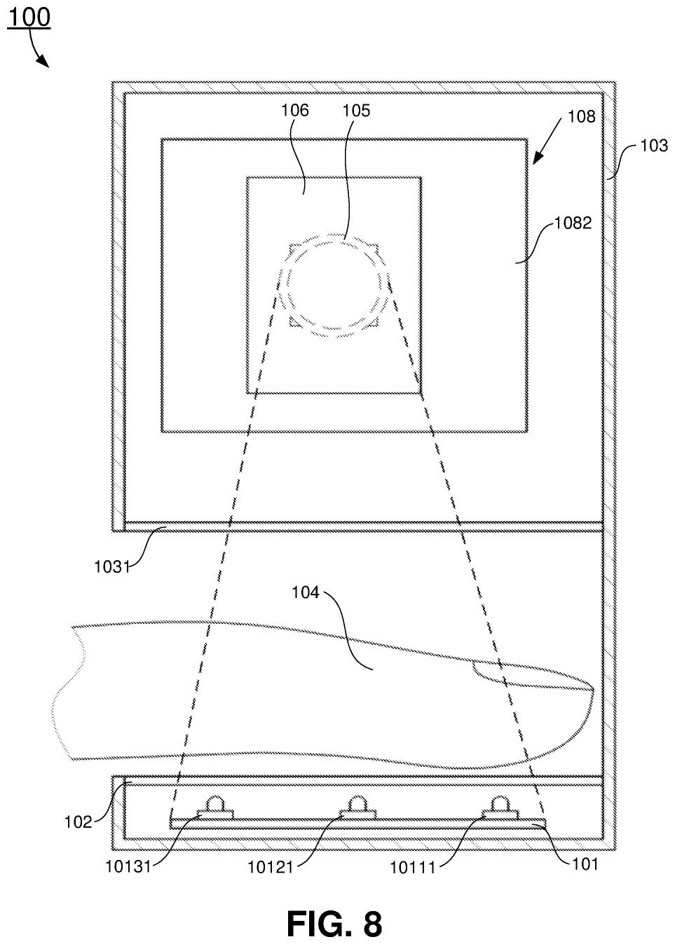

In certain embodiments, the infrared light source 101 includes a group of infrared light-emitting diodes (LED), a group of infrared light bulbs, and/or any other infrared light sources. In certain embodiments, in order to generate better quality of finger vein pattern images, the group of infrared LED and the group of infrared light bulbs are arranged in a matrix form having N rows 101N1, 101N2, . . . , and 101NM, and M columns 1011M, 1012M, . . . , and 101NM, as shown in FIG. 3-FIG. 8. In one embodiment, all N×M infrared LEDs can be lit up to generate finger vein pattern images.

In another embodiment, some of N×M infrared LEDs can be lit up to generate finger vein pattern images. In an additional embodiment, each of the N rows of infrared LEDs can be lit up in turn to generate a scanning infrared light source 101 vertically. In yet another embodiment, each of the M column of infrared LEDs can be lit up in turn to generate another scanning infrared light source 101 horizontally. These variations of lighting patterns are created to alter parameters of infrared light source 101 such as LED light intensity, lighting directions, and lighting orientations to generate better quality finger vein pattern images.

In certain embodiments, the finger vein sensor controller 200 includes a processor 202, and a non-volatile memory 204. The non-volatile memory 204 stores an operating system 2042 and computer executable instructions 2044. The computer executable instructions 2044 include: an infrared light source control module 20441, a finger vein verification module 20442, an image/parameter storage module 20443, an image processing module 20444, an image sensor control module 20445, and a finger vein sensor power module 20446. The infrared light source control module 20441 controls the infrared light source 101, creates various infrared lighting patterns to alter the parameters of infrared light source 101 such as LED light intensity, lighting directions, and lighting orientations to generate better quality finger vein pattern images. The finger vein verification module 20442 verifies identity of the target human using the finger vein pattern images captured by the image sensor 106. The image/parameter storage module 20443 stores the finger vein pattern images captured by the image sensor 106, as well as various parameters of the infrared light source 101. The image processing module 20444 processes the captured finger vein pattern images, and based on the processing results, the lighting patterns of the infrared light source 101 is changed through the infrared light source control module 20441 to generate improved image quality of the finger vein pattern images. The image sensor control module 20445 controls exposure sensitivity, exposure timing and exposure sequence of the image sensor 106 to capture improved image quality of the finger vein pattern images.

In certain embodiments, the finger vein sensor power module 20446 provides electrical power to the finger vein sensor 100 for supporting the operation of the finger vein sensor 100. In one embodiment, the finger vein sensor power module 20446 may receive electrical power from an external source, such as an alternate current (AC) electrical source, or a direct current (DC) electrical source. In another embodiment, the finger vein sensor power module 20446 may receive electrical power from a battery. In yet another embodiment, the finger vein sensor power module 20446 may receive electrical power from a rechargeable battery. The rechargeable battery includes at least one of: a lead-acid rechargeable battery, a nickel cadmium (NiCd) rechargeable battery, a nickel metal hydride (NiMH) rechargeable battery, a lithium ion (Li-ion) rechargeable battery, and a lithium ion polymer (Li-ion polymer) rechargeable battery.

In certain embodiments, when executed at the processor 202, the computer executable instructions 2044 cause the processor 202 to perform one or more of operations:

detecting, by the image sensor 106, that the finger 104 is placed between the infrared light source 101 and the image sensor 106;

irradiating, by the infrared light source 101 from the lower end of the finger vein sensor 100, the infrared light through the finger 104 to generate an infrared image of finger vein pattern of the finger 104 on the image sensor 106;

detecting, by the image sensor 106, clarity, brightness, contrast, noise distortion and blurring distortion of the finger vein pattern image of the finger 104 of the target human;

adjusting, by the infrared light source control module 20441, a set of parameters of the infrared light source 101 such as LED light intensity, lighting directions, and lighting orientations, until each of the clarity, the brightness, the contrast, the noise distortion, and the blurring distortion of the finger vein pattern image of the finger 104 reaches a corresponding predetermined level; and

capturing, by the image sensor 106, the infrared image of finger vein pattern of the finger 104, and storing the captured infrared image of finger vein pattern of the finger 104 into the image/parameter storage module 20443.

In certain embodiments, the finger vein sensor 100 may also include a finger vein sensor enclosure 103 as shown in FIG. 1. The finger vein sensor enclosure 103 includes a lower compartment, and an upper compartment. The infrared light source 101 is positioned in the lower compartment and provides infrared light through a transparent finger resting surface 102 vertically from the lower compartment.

A lens 105, an infrared filter 107 and the image sensor 106 are positioned in the upper compartment. A lower surface 1031 of the upper compartment is a transparent surface to allow the image sensor 106 to capture the finger vein pattern image of the finger 104 formed above the transparent finger resting surface 102. This transparent finger resting surface 102 allows the target human to rest the finger 104 on it and generates a steady finger vein pattern image at a fixed location. It prevents inconsistency when the finger 104 moves up and down.

The lens 105 is positioned between the finger 104 and the image sensor 106. The lens 105 is adjusted to focus on the finger vein pattern generated above the transparent finger resting surface 102. The combination of the lens 105 and the transparent finger resting surface 102 allows the image sensor 106 to capture consistent finger vein pattern images and to improve quality of the infrared image of finger vein pattern of the finger 104.

In certain embodiments, the infrared filter 107 is positioned between the lens 105 and the image sensor 106 for improving quality of the infrared image of finger vein pattern of the finger 104. The infrared filter 107 allows infrared lights to pass and eliminates light interference from any lights other than infrared light. Therefore, the application of the infrared filter 107 also improves the quality of the infrared image of finger vein pattern of the finger 104.

The configuration of the finger vein sensor 100 creates larger size finger vein pattern images than the conventional finger vein sensors. Conventional finger vein sensor allows user to touch the image forming surface of the finger vein sensor, any contamination on the image forming surface of the finger vein sensor will be captured by the conventional vein sensor and it will cause authentication errors. The finger vein sensor 100 prevents such errors from happening. Any contaminations such as dirt on the surface of the finger 104, the dirt accumulated on the transparent finger resting surface 102, or finger prints left on the transparent finger resting surface 102 will not be captured by the image sensor 106. The water stains or water accumulated on the transparent finger resting surface 102 will not cause any authentication errors because the water will be transparent and will not distort the finger vein pattern of the finger 104.

As shown in FIG. 1, in order to create large finger vein pattern image, the image sensor 106 should have sufficient distance from the finger 104 resting on the transparent finger resting surface 102 because of the straight infrared light path from the bottom to the top of the finger vein sensor enclosure 103. This may cause the finger vein sensor enclosure 103 to become tall. In order to shorten the height of the finger vein sensor enclosure 103, a few more exemplary embodiments of finger vein sensors are described as following. In certain embodiments, the straight infrared light path from the bottom to the top of the finger vein sensor enclosure 103 may be reflected by an optical reflector to become a horizontal infrared light path.

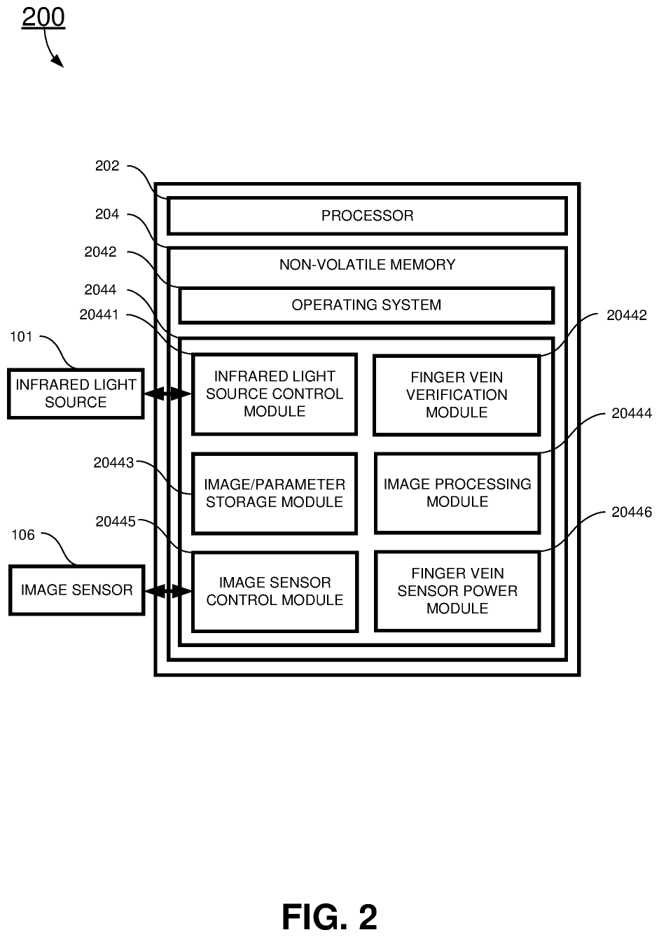

Referring now to FIGS. 2A through AD, in another aspect, the present disclosure relates to a finger vein sensor 102. In certain embodiments, the finger vein sensor 102 includes: an image sensor 1026, an infrared light source 1021, and an optical reflector 1028.

In certain embodiments, the infrared light source 1021 may include includes a predetermined number of infrared light-emitting diodes (LEDs), a predetermined number of infrared light bulbs, and/or any other infrared light sources. As shown in the side view of the finger vein sensor 102, the infrared light source 1021 includes a first infrared LED 10211, a second infrared LED 10212, and a third infrared LED 10213. The infrared LED or the infrared light bulbs may be arranged in one or more rows and one or more columns and positioned at the bottom of the finger vein sensor 102. These three LEDs as shown in FIGS. 1A and 1B form one column of the infrared light source 1021. The infrared light source 1021 may include more than one columns of infrared LEDs (not shown in FIGS. 2A through 2D). Such infrared light source 1021 provides high intensity and evenly distributed infrared light to penetrate a finger 1024 of a target human and generates large size finger vein pattern images with more clarity.

In certain embodiments, the image sensor 1026 captures at least one infrared image of finger vein pattern of the finger 1024. The image sensor 1026 faces the target human in a horizontal direction and is positioned at the top of the finger vein sensor 102. The finger 1024 is positioned between the infrared light source 1021 and the image sensor 1026. The infrared light from the infrared light source 1021 irradiates the finger 1024 vertically from the bottom to generate the infrared image of finger vein pattern of the finger 1024. The optical reflector 1028 includes a reflecting mirror 10281 as shown in FIGS. 2A and 2B, a triangular reflecting glass 10282 as shown in FIGS. 2C and 2D, or any other optical reflecting devices (not shown in FIGS. 2A through 2D). The optical reflector 1028 is positioned between the image sensor 1026 and the infrared light source 1021 for reflecting the vertically oriented infrared image of finger vein pattern of the finger 1024 to the horizontally oriented image sensor 1026.

In certain embodiments, the finger 1024 is positioned between the infrared light source 1021 and the image sensor 1026, the infrared light from the infrared light source 1021 irradiates the finger 1024 vertically from the bottom to generate the infrared image of finger vein pattern of the finger 1024 in a vertical direction, and the infrared image of finger vein pattern of the finger 1024 is reflected by the optical reflector 1028 and captured by the horizontally oriented image sensor 1026.

In certain embodiments, the finger vein sensor 102 may include a transparent finger resting surface 1022 for resting the finger 1024. This transparent finger resting surface 1022 allows the user to rest the finger 1024 on it and generates a steady finger vein pattern image at a fixed location. It prevents inconsistency when the finger 1024 moves up and down.

In certain embodiments, the finger vein sensor 102 may also include a lens 1025. The lens 1025 is positioned between the finger 1024 and the image sensor 1026. The lens 1025 is adjusted to focus on the finger vein pattern generated above the transparent finger resting surface 1022. The combination of the lens 1025 and the transparent finger resting surface 1022 allows the image sensor 1026 to capture consistent finger vein pattern images and improve quality of the infrared image of finger vein pattern of the finger 1024.

In certain embodiments, the finger vein sensor 102 may also include an infrared filter 1027. The infrared filter 1027 may be placed between the lens 1025 and the image sensor 1026. The infrared filter 1027 allows infrared lights to pass and eliminates light interference from any lights other than infrared light. Therefore, the application of the infrared filter 1027 also improves the quality of the infrared image of finger vein pattern of the finger 1024.

In certain embodiments, the finger vein sensor 102 may also include a finger vein sensor enclosure 1023 as shown in FIGS. 2B and 2D. The finger vein sensor enclosure 1023 includes a lower compartment, and an upper compartment. The infrared light source 1021 is positioned in the lower compartment and provides infrared light through the transparent finger resting surface 1022 vertically from the lower compartment. The lens 1025, the infrared filter 1027 and the image sensor 1026 are positioned horizontally in the upper compartment. A lower surface 10231 of the upper compartment is a transparent surface to allow the image sensor 1026 to capture the finger vein pattern image of the finger 1024 formed above the transparent finger resting surface 1022. The vertical finger vein pattern image of the finger 1024 is reflected by the optical reflector 1028 and turned to a horizontal finger vein pattern image of the finger 1024 to be captured by the horizontally oriented image sensor 1026.

In additional to have the horizontally oriented image sensor 1026 facing the target human, the vertically generated finger vein pattern of the finger 1024 may be reflected by the optical reflector 1028 and captured by the horizontally oriented image sensor 1026 positioned on either side of the finger vein sensor 102. Referring now to FIGS. 3A through 3H, in yet another aspect, the present disclosure relates to a finger vein sensor 104. In certain embodiments, the finger vein sensor 104 includes: an image sensor 1046, an infrared light source 1041, and an optical reflector 1048. The image sensor 1046 captures at least one infrared image of finger vein pattern of a finger 1044 of a target human. In one embodiment, the image sensor 1046 is positioned on the top right side of the finger vein sensor 104 facing the center of the finger vein sensor 104 in a horizontal direction. In another embodiment, the image sensor 1046 is positioned on the top left side of the finger vein sensor 104 facing the center of the finger vein sensor 104 in a horizontal direction.

In certain embodiments, the infrared light source 1041 may include includes a predetermined number of infrared light-emitting diodes (LEDs), a predetermined number of infrared light bulbs, and/or any other infrared light sources. As shown in the front view of the finger vein sensor 104, the infrared light source 1041 includes a first infrared LED 10411, a second infrared LED 10412, and a third infrared LED 10413. As shown in the side view of the finger vein sensor 104, the infrared light source 1041 includes the third infrared LED 10413, a fourth infrared LED 10414, and a fifth infrared LED 10415. In the exemplary embodiment shown in FIGS. 3A and 3B, the infrared LED or the infrared light bulbs may be arranged in three rows and three columns and positioned at the bottom of the finger vein sensor 104. Such infrared light source 1041 provides high intensity and evenly distributed infrared light to penetrate the finger 1044 of the target human and generates large size finger vein pattern images with more clarity.

In certain embodiments, the optical reflector 1048 includes a reflecting mirror 10481, as shown in FIGS. 3A through 3D, a triangular reflecting glass 10482, as shown in FIGS. 3E through 3H, or any other optical reflecting devices (not shown in FIGS. 3A through 3H). The optical reflector 1048 is positioned between the image sensor 1046 and the infrared light source 1041 for reflecting the vertically oriented infrared image of finger vein pattern of the finger 1044 to the horizontally oriented image sensor 1046.

In certain embodiments, the finger 1044 is positioned between the infrared light source 1041 and the image sensor 1046, the infrared light from the infrared light source 1041 irradiates the finger 1044 vertically from the bottom to generate the infrared image of finger vein pattern of the finger 1044 in a vertical direction, and the vertically oriented infrared image of finger vein pattern of the finger 1044 is reflected by the optical reflector 1048 and captured by the horizontally oriented image sensor 1046.

In certain embodiments, the finger vein sensor 104 may include a transparent finger resting surface 1042 for resting the finger 1044. This transparent finger resting surface 1042 allows the user to rest the finger 1044 on it and generates a steady finger vein pattern image at a fixed location. It prevents inconsistency when the finger 1044 moves up and down.

In certain embodiments, the finger vein sensor 104 may also include a lens 1045. The lens 1045 is positioned between the finger 1044 and the image sensor 1046. The lens 1045 is adjusted to focus on the finger vein pattern generated above the transparent finger resting surface 1042. The combination of the lens 1045 and the transparent finger resting surface 1042 allows the image sensor 1046 to capture consistent finger vein pattern images and improve quality of the infrared image of finger vein pattern of the finger 1044.

In certain embodiments, the finger vein sensor 104 may also include an infrared filter 1047. The infrared filter 1047 may be placed between the lens 1045 and the image sensor 1046. The infrared filter 1047 allows infrared lights to pass and eliminates light interference from any lights other than infrared light. Therefore, the application of the infrared filter 1047 also improves the quality of the infrared image of finger vein pattern of the finger 1044.

In certain embodiments, the finger vein sensor 104 may also include a finger vein sensor enclosure 1043 as shown in FIGS. 3C, 3D, 3G, and 3H. The finger vein sensor enclosure 1043 includes a lower compartment, and an upper compartment. The infrared light source 1041 is positioned in the lower compartment and provides infrared light through the transparent finger resting surface 1042 vertically from the lower compartment. The lens 1045, the infrared filter 1047 and the image sensor 1046 are positioned horizontally in the upper compartment. A lower surface 10431 of the upper compartment is a transparent surface to allow the image sensor 1046 to capture the finger vein pattern image of the finger 1044 formed above the transparent finger resting surface 1042. The vertical finger vein pattern image of the finger 1044 is reflected by the optical reflector 1048 and turned to a horizontal finger vein pattern image of the finger 1044 to be captured by the horizontally oriented image sensor 1046.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope. Accordingly, the scope of the present disclosure is defined by the appended claims rather than the foregoing description and the exemplary embodiments described therein.

What is claimed is:

1. A finger vein sensor comprising:
an image sensor horizontally positioned at an upper end of the finger vein sensor, wherein the image sensor faces down in vertical direction to capture at least one infrared image of finger vein pattern of a finger of a target human, and
an infrared light source positioned at a lower end of the finger vein sensor, wherein the infrared light source faces up and shines infrared light upward in vertical direction; and
a finger vein sensor controller, wherein the finger vein sensor controller comprises a processor, and a non-volatile memory storing an operating system and computer executable instructions, wherein the computer executable instructions comprise: an infrared light source control module, a finger vein verification module, an image/parameter storage module, an image processing module, an image sensor control module, and a finger vein sensor power module, wherein when executed at the processor, the computer executable instructions cause the processor to perform one or more of operations:
detecting, by the image sensor, that the finger is placed between the infrared light source and the image sensor;
irradiating, by the infrared light source from the lower end of the finger vein sensor, the infrared light through the finger to generate an infrared image of finger vein pattern of the finger on the image sensor;
detecting, by the image sensor, clarity, brightness, contrast, noise distortion and blurring distortion of the finger vein pattern image of the finger of the target human;
adjusting, by the infrared light source control module, a plurality of parameters of the infrared light source, until each of the clarity, the brightness, the contrast, the noise distortion, and the blurring distortion of the finger vein pattern image of the finger reaches a corresponding predetermined level; and
capturing, by the image sensor, the infrared image of finger vein pattern of the finger, and storing the captured infrared image of finger vein pattern of the finger into the image/parameter storage module.

2. The finger vein sensor of claim 1, wherein the infrared light source comprises a plurality of infrared light-emitting diodes (LED), a plurality of infrared light bulbs, and/or any other infrared light sources, wherein the plurality of infrared LED and the plurality of infrared light bulbs are arranged in one or more rows and one or more columns.

3. The finger vein sensor of claim 1, further comprising a lens positioned between the finger and the image sensor, and an infrared filter positioned between the lens and the image sensor for improving quality of the infrared image of finger vein pattern of the finger.

4. The finger vein sensor of claim 3, further comprising a transparent finger resting surface for resting the finger on the transparent finger resting surface.

5. The finger vein sensor of claim 4, further comprising a finger vein sensor enclosure having a lower compartment for positioning the infrared light source and an upper compartment for positioning the image sensor and the lens.

6. The finger vein sensor of claim 5, wherein an upper surface of the lower compartment forms the transparent finger resting surface, and a lower surface of the upper compartment forms a transparent surface.

7. A finger vein sensor comprising:
an image sensor vertically positioned in a user-facing side of an upper end of the finger vein sensor, wherein the image sensor faces center of the finger vein sensor in horizontal direction to capture at least one infrared image of finger vein pattern of a finger of a target human;
an infrared light source positioned at a lower end of the finger vein sensor, wherein the infrared light source faces upward and shines infrared light upward in vertical direction;
an optical reflector positioned in an optical path between the image sensor and the infrared light source, wherein the optical reflector reflects a vertically oriented infrared image of finger vein pattern of the finger to the horizontally oriented image sensor; and
a finger vein sensor controller, wherein the finger vein sensor controller comprises a processor, and a non-volatile memory storing an operating system and computer executable instructions, wherein the computer executable instructions comprise: an infrared light source control module, a finger vein verification module, an image/parameter storage module, an image processing module, an image sensor control module, and a finger vein sensor power module, wherein when executed at the processor, the computer executable instructions cause the processor to perform one or more of operations:
detecting, by the image sensor, that the finger is placed between the infrared light source and the optical reflector;
irradiating, by the infrared light source from the lower end of the finger vein sensor, the infrared light through the finger to generate an infrared image of finger vein pattern of the finger on the image sensor through the optical reflector;
detecting, by the image sensor, clarity, brightness, contrast, noise distortion and blurring distortion of the finger vein pattern image of the finger of the target human;
adjusting, by the infrared light source control module, a plurality of parameters of the infrared light source, until each of the clarity, the brightness, the contrast, the noise distortion, and the blurring distortion of the finger vein pattern image of the finger reaches a corresponding predetermined level; and
capturing, by the image sensor, the infrared image of finger vein pattern of the finger, and storing the captured infrared image of finger vein pattern of the finger into the image/parameter storage module.

8. The finger vein sensor of claim 7, wherein the infrared light source comprises a plurality of infrared light-emitting diodes (LED), a plurality of infrared light bulbs, and/or any other infrared light sources, wherein the plurality of infrared LED and the plurality of infrared light bulbs are arranged in one or more rows and one or more columns.

9. The finger vein sensor of claim 7, wherein the optical reflector comprises a reflecting mirror 1081, a triangular reflecting glass, or any other optical reflecting devices.

10. The finger vein sensor of claim 7, wherein further comprising a lens positioned between the optical reflector and the image sensor, and an infrared filter positioned between the lens and the image sensor for improving quality of the infrared image of finger vein pattern of the finger.

11. The finger vein sensor of claim 10, further comprising a transparent finger resting surface such that the finger is rested on the transparent finger resting surface.

12. The finger vein sensor of claim 11, further comprising a finger vein sensor enclosure having a lower compartment for positioning the infrared light source and an upper compartment for positioning the image sensor and the lens.

13. The finger vein sensor of claim 12, wherein an upper surface of the lower compartment forms the transparent finger resting surface, and a lower surface of the upper compartment forms a transparent surface.

14. A finger vein sensor comprising:
an image sensor, wherein the image sensor is positioned in an upper end of the finger vein sensor to capture at least one infrared image of finger vein pattern of a finger of a target human;
an infrared light source positioned at a lower end of the finger vein sensor;
wherein the infrared light source faces upward and shines infrared light upward in vertical direction;
an optical reflector positioned in an optical path between the image sensor and the infrared light source, wherein the optical reflector reflects a vertically oriented infrared image of finger vein pattern of the finger to the horizontally oriented image sensor; and
a finger vein sensor controller, wherein the finger vein sensor controller comprises a processor, and a non-volatile memory storing an operating system and computer executable instructions, wherein the computer executable instructions comprise: an infrared light source control module, a finger vein verification module, an image/parameter storage module, an image processing module, an image sensor control module, and a finger vein sensor power module, wherein when executed at the processor, the computer executable instructions cause the processor to perform one or more of operations:
detecting, by the image sensor, that the finger is placed between the infrared light source and the optical reflector;
irradiating, by the infrared light source from the lower end of the finger vein sensor, the infrared light through the finger to generate an infrared image of finger vein pattern of the finger on the image sensor through the optical reflector;
detecting, by the image sensor, clarity, brightness, contrast, noise distortion and blurring distortion of the finger vein pattern image of the finger of the target human;
adjusting, by the infrared light source control module, a plurality of parameters of the infrared light source, until each of the clarity, the brightness, the contrast, the noise distortion, and the blurring distortion of the finger vein pattern image of the finger reaches a corresponding predetermined level; and
capturing, by the image sensor, the infrared image of finger vein pattern of the finger, and storing the captured infrared image of finger vein pattern of the finger into the image/parameter storage module.

15. The finger vein sensor of claim 14, wherein the image sensor is vertically positioned in a left side of the upper end of the finger vein sensor, wherein the image sensor faces a center of the finger vein sensor in horizontal direction to capture the at least one infrared image of finger vein pattern of the finger of the target human through the optical reflector.

16. The finger vein sensor of claim 14, wherein the image sensor is vertically positioned in a right side of the upper end of the finger vein sensor, wherein the image sensor faces a center of the finger vein sensor in horizontal direction to capture the at least one infrared image of finger vein pattern of the finger of the target human through the optical reflector.

17. The finger vein sensor of claim 14, wherein the infrared light source comprises a plurality of infrared light-emitting diodes (LED), a plurality of infrared light bulbs, and/or any other infrared light sources, wherein the plurality of infrared LED and the plurality of infrared light bulbs are arranged in one or more rows and one or more columns.

18. The finger vein sensor of claim 14, wherein the optical reflector comprises a reflecting mirror 1081, a triangular reflecting glass, or any other optical reflecting devices.

19. The finger vein sensor of claim 14, further comprising:
a lens positioned between the optical reflector and the image sensor; and
an infrared filter positioned between the lens and the image sensor for improving quality of the infrared image of finger vein pattern of the finger.

20. The finger vein sensor of claim 19, further comprising: a finger vein sensor enclosure having a lower compartment for positioning the infrared light source and an upper compartment for positioning the image sensor and the lens, wherein an upper surface of the lower compartment forms a transparent finger resting surface, and a lower surface of the upper compartment forms a transparent surface.
